SEQUENCE LISTING (12 sequences)

SEQ ID NO: 1 - DNA, 2229 bp, Homo sapiens
atgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcctcgacgaggccttccagcgctatcgtgacctgcttttcggttccgggtcttggccccgtccttacctcacagggaaacggcatacactggagaagaatgtgttggttgtctctgtagtcacacctggatgtaaccagcttcctactttggagtcagtggagaattataccctgaccataaatgatgaccagtgtttactcctctctgagactgtctggggagctctccgaggtctggagacttttagccagcttgtttggaaatctgctgagggcacattctttatcaacaagactgagattgaggactttccccgctttcctcaccggggcttgctgttggatacatctcgccattacctgccactctctagcatcctggacactctggatgtcatggcgtacaataaattgaacgtgttccactggcatctggtagatgatccttccttcccatctgagagcttcacttttccagagctcatgagaaaggggtcctacaaccctgtcacccacatctacacagcacaggatgtgaaggaggtcattgaatacgcacggctccggggtatccgtgtgcttgcagagtttgacactcctggccacactttgtcctggggaccaggtatccctggattactgactccttgctactctgggtctgagccctctggcacctttggaccagtgaatcccagtctcaataatacctatgagttcatgagcacattcttcttagaagtcagctctgtcttcccagatttttatcttcatcttggaggagatgaggttgatttcacctgctggaagtccaacccagagatccaggactttatgaggaagaaaggcttcggtgaggacttcaagcagctggagtccttctacatccagacgctgctggacatcgtctcttcttatggcaagggctatgtggtgtggcaggaggtgtttgataataaagtaaagattcagccagacacaatcatacaggtgtggcgagaggatattccagtgaactatatgaaggagctggaactggtcaccaaggccggcttccgggcccttctctctgccccctggtacctgaaccgtatatcctatggccctgactggaaggatttctacgtagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgagttgctgaggcgaggtgtccaggcccaacccctcaatgtaggcttctgtgagcaggagtttgaacagacctgagccccaggcaccgaggagggtgctggctgtaggtgaatggtagtggagccaggcttccactgcatcctggccaggggacggagccccttgccttcgtgccccttgcctgcgtgcccctgtgcttggagagaaaggggccggtgctggcgctcgcattcaataaagagtaatgtggcatttttctataataaacatggattacctgtgtttaaaaaaaaaagtgtgaatggcgttagggtaagggcacagccaggctggagtcagtgtctgcccctgaggtcttttaagttgagggctgggaatgaaacctatagcctttgtgctgttctgccttgcctgtgagctatgtcactcccctcccactcctgaccatattccagacacctgccctaatcctcagcctgctcacttcacttctgcattatatctccaaggcgttggtatatggaaaaagatgtaggggcttggaggtgttctggacagtggggagggctccagacccaacctggtcacaaaagagcctctcccccatgcatactcatccacctccctcccctagagctattctcctttgggtttcttgctgctgcaattttatacaaccattatttaaatattattaaacacatattgttctct

SEQ ID NO: 2 - DNA, 2229 bp, Homo sapiens
atgacaagctccaggctttggttttcgctgctgctggcggcagcgttcgcaggacgggcgacggccctctggccctggcctcagaacttccaaacctccgaccagcgctacgtcctttacccgaacaactttcaattccagtacgatgtcagctcggccgcgcagcccggctgctcagtcttcgacgaggccttccagcgctatcgtgacctgcttttcggttccgggtcttggccccgtccttacctcacagggaaacggcatacactggagaagaatgtgttggttgtctctgtagtcacacctggatgtaaccagcttcctactttggagtcagtggagaattataccctgaccataaatgatgaccagtgtttactcctctctgagactgtctggggagctctccgaggtctggagacttttagccagcttgtttggaaatctgctgagggcacattctttatcaacaagactgagattgaggactttccccgctttcctcaccggggcttgctgttggatacatctcgccattacctgccactctctagcatcctggacactctggatgtcatggcgtacaataaattgaacgtgttccactggcatctggtagatgatccttccttcccatatgagagcttcacttttccagagctcatgagaaaggggtcctacaaccctgtcacccacatctacacagcacaggatgtgaaggaggtcattgaatacgcacggctccggggtatccgtgtgcttgcagagtttgacactcctggccacactttgtcctggggaccaggtatccctggattactgactccttgctactctgggtctgagccctctggcacctttggaccagtgaatcccagtctcaataatacctatgagttcatgagcacattcttcttagaagtcagctctgtcttcccagatttttatcttcatcttggaggagatgaggttgatttcacctgctggaagtccaacccagagatccaggactttatgaggaagaaaggcttcggtgaggacttcaagcagctggagtccttctacatccagacgctgctggacatcgtctcttcttatggcaagggctatgtggtgtggcaggaggtgtttgataataaagtaaagattcagccagacacaatcatacaggtgtggcgagaggatattccagtgaactatatgaaggagctggaactggtcaccaaggccggcttccgggcccttctctctgccccctggtacctgaaccgtatatcctatggccctgactggaaggatttctacgtagtggaacccctggcatttgaaggtacccctgagcagaaggctctggtgattggtggagaggcttgtatgtggggagaatatgtggacaacacaaacctggtccccaggctctggcccagagcaggggctgttgccgaaaggctgtggagcaacaagttgacatctgacctgacatttgcctatgaacgtttgtcacacttccgctgtgagttgctgaggcgaggtgtccaggcccaacccctcaatgtaggcttctgtgagcaggagtttgaacagacctgagccccaggcaccgaggagggtgctggctgtaggtgaatggtagtggagccaggcttccactgcatcctggccaggggacggagccccttgccttcgtgccccttgcctgcgtgcccctgtgcttggagagaaaggggccggtgctggcgctcgcattcaataaagagtaatgtggcatttttctataataaacatggattacctgtgtttaaaaaaaaaagtgtgaatggcgttagggtaagggcacagccaggctggagtcagtgtctgcccctgaggtcttttaagttgagggctgggaatgaaacctatagcctttgtgctgttctgccttgcctgtgagctatgtcactcccctcccactcctgaccatattccagacacctgccctaatcctcagcctgctcacttcacttctgcattatatctccaaggcgttggtatatggaaaaagatgtaggggcttggaggtgttctggacagtggggagggctccagacccaacctggtcacaaaagagcctctcccccatgcatactcatccacctccctcccctagagctattctcctttgggtttcttgctgctgcaattttatacaaccattatttaaatattattaaacacatattgttctct

SEQ ID NO: 3 - DNA, 21 bp, Homo sapiens
ttccaaacctccgaccagcgc

SEQ ID NO: 4 - DNA, 21 bp, Homo sapiens
cagagtgtccaggattgctag

SEQ ID NO: 5 - DNA, 25 bp, Homo sapiens
ttctttatcaacaagactgagattg

SEQ ID NO: 6 - DNA, 27 bp, Homo sapiens
gcaggtgaaatcaacctcatctcctcc

SEQ ID NO: 7 - DNA, 27 bp, Homo sapiens
atccctggattactgactccttgctac

SEQ ID NO: 8 - DNA, 21 bp, Homo sapiens
cttcaaatgccaggggttcca

SEQ ID NO: 9 - DNA, 20 bp, Homo sapiens
agccagacacaatcatacag

SEQ ID NO: 10 - DNA, 20 bp, Homo sapiens
cctttctctccaagcacagg

SEQ ID NO: 11 - DNA, 33 bp, Homo sapiens
aaaagtgaagctctcagatgggaaggaaggatc

SEQ ID NO: 12 - DNA, 18 bp, Homo sapiens
ctcgtcgaagactgagca

(19) United States
(12) Patent Application Publication
(10) Pub. No.: US 2007/0218469 A1
(43) Pub. Date: Sep. 20, 2007

(54) Novel mutations in hexosaminidase A

(76) Inventor: Ruth Navon, Ramat Gan (IL)

Correspondence Address: NATH & ASSOCIATES, PLLC, 112 South West Street, Alexandaria, VA, 22314, US

(21) Appl. No.: 11/541,568

(22) Filed: Oct. 3, 2006

Related U.S. Application Data
(60) Provisional application No. 60/722,427, filed on Oct. 3, 2005.

(51) Int. Cl. A61K 48/00; C12Q 1/68; C07H 21/04

(52) U.S. Cl. 435/6; 536/24.3; 435/320.1; 435/325; 514/44

(57) ABSTRACT

The present invention discloses methods and compositions for detecting novel mutations in the α chain of the hexosaminidase gene. These methods facilitate rapid screening for Tay-Sachs disease carriers, especially in the Ashkenazi Jewish population. The novel mutations include the single nucleotide substitutions 638A>C and 181C>T.

This application claims the benefit of prior U.S. provisional patent application No. 60/722,427 filed Oct. 3, 2005, the contents of which are hereby incorporated by reference in their entirety.

### FIELD OF THE INVENTION

This invention relates to the identification of mutations in the α chain of the lysosomal enzyme β-N-acetyl-hexosaminidase A. More particularly, the present invention concerns nucleic acid compositions and kits useful for screening, diagnosis and prognosis of a genetic defect which is associated with Tay Sachs disease.

### BACKGROUND OF THE INVENTION

Two major isozymes of the lysosomal enzyme β-N-acetyl-hexosaminidase (Hex) (EC 3.2.1.52) exist: Hex A, a heterodimer of α and β subunits, and Hex B, a homodimer of the β subunits. Hex A almost exclusively degrades the substrate G_{M2} ganglioside in the presence of an activator protein. Hex A deficiency, caused by mutations in the HEXA gene encoding the α subunit, results in accumulation of G_{M2} ganglioside, mainly in neuronal tissues and manifests in various progressive neurological syndromes termed G_{M2} gangliosidosis type B (Gravel et al 2001). The degree of G_{M2} ganglioside accumulation is directly correlated with the level of Hex A deficiency; thus, mutations that result in total absence of Hex A activity lead to Tay-Sachs disease (TSD), the severe-infantile form of G_{M2} gangliosidosis type B, whereas mutations that result in residual Hex A activity give rise to the milder juvenile and the late onset of TSD (LOTS) (MIM #272800). Possessing a residual level of activity more than 5% of the normal Hex A level was suggested as the “critical threshold” that allows one to escape the severe TSD, and exceeding a level of 10%, to escape the chronic LOTS form (Conzelmann et al 1983; Conzelmann and Sandhoff 1983-4).

To date, approximately 100 mutations, including neutral genetic polymorphisms, have been reported in the HEXA gene (Myerowitz, 1997; U.S. Pat. No. 5,217,865). Some of the mutations have been thoroughly characterized and a correlation between genotype and phenotype severity can be foreseen. However, there is no obvious correlation between genotypes and phenotypes of the neurological condition, especially in the non-infantile form of TSD. Although almost all LOTS patients carry the missense mutation G269S in compound heterozygosity with another missense mutation (Navon and Proia 1989; Paw et al 1989), they display different degrees of severity, and even patients with identical mutations manifest marked clinical heterogeneity (Argov and Navon 1984; Navon 1991).

U.S. Pat. No. 5,217,865 discloses methods for detecting mutations in the HEXA gene for diagnosis of Tay-Sachs disease patients and carriers. The patent describes a splice junction mutation and an insertion mutation, but does not disclose the novel mutations of the present invention.

### SUMMARY OF THE INVENTION

The present invention describes newly identified point mutations in the α chain of beta-hexosaminidase (hereinafter HEXA) which are associated with Tay Sachs disease.

One such point mutation occurs at position 638 wherein Adenine is replaced with Cytosine (hereinafter 638A>C).

Another point mutation occurs at position 181, wherein Cytosine is replaced with Thymine (hereinafter 181C>T).

In a first of its aspects, the present invention provides a method to determine the presence or absence of an HEXA gene mutation in an individual comprising: isolating a nucleic acid sample being genomic DNA, cDNA, or RNA from an individual, and assessing the presence or absence of an allele carrying the mutation; wherein said mutation is either 638A>C or 181C>T.

The presence of two mutation-carrying alleles indicates that the individual is affected with Tay Sachs disease. The presence of one mutation-carrying allele indicates that the individual is either a heterozygote carrier of Tay Sachs disease or is affected with Tay Sachs disease depending on the identification of additional known mutations in the individual's second allele. Such additional mutations are well documented in the art, e.g. Myerowitz, 1997, U.S. Pat. No. 5,217,865.
In one embodiment, the assessing step is performed by a process comprising: (a) subjecting the nucleic acid molecules in the sample to amplification using oligonucleotide primers specific for the HEXA gene; (b) hybridizing the amplification product with a nucleotide probe specific for the mutation (c) measuring the intensity of hybridization.

In one embodiment the nucleotide probe specific for the 638A>C mutation is 5′AAAAGTGAAGCTCTCAGATGGGAAGGAAGGATC3′ (SEQ ID. No 11).

In another embodiment, the nucleotide probe specific for the 181C>T mutation is 5′CTCGTCGAAGACTGAGCA3′ (SEQ ID. No 12).

The invention further provides a method for screening or diagnosing patients or carriers of Tay Sachs disease comprising detecting said 181C>T or 638A>C mutation in said patients' genetic material.

In a preferred embodiment the invention allows for screening or diagnosing patients or carriers of Tay Sachs disease in the Ashkenazi Jewish population comprising detecting said 181C>T mutation in said patients' genome.

In another aspect the present invention provides a kit for detecting the 181C>T or 638A>C mutation, useful for screening the population or for diagnosing Tay Sachs disease or carrier status.

The invention further provides isolated nucleic acid sequences corresponding to the HEXA gene (SEQ ID NO. 1), wherein the Adenine nucleotide at position 638 is replaced with Cytosine, or portions thereof comprising the 638A>C mutation and its flanking regions.

The invention also provides isolated nucleic acid sequences corresponding to the HEXA gene (SEQ ID NO. 2), wherein the Cytosine nucleotide at position 181 is replaced with Thymine, or portions thereof comprising the 181C>T mutation and its flanking regions.

In accordance with another aspect, the present invention provides nucleic acid probes, at least 18 nucleotides ling, comprising a nucleotide sequence complementary to the nucleic acid sequences as set forth above.

The present invention further provides an expression vector comprising a coding sequence of a nucleic acid as set forth above operably linked with a promoter sequence capable of directing expression of the coding sequence in host cells for the vector.

In accordance with another aspect, the present invention provides host cells transformed with a vector as set forth above.

Another aspect of the present invention is a method of treating Tay-Sachs disease by gene therapy based on homologous recombination using corrective vectors. The method comprising: (a) providing corrective recombinant vectors comprising a nucleic acid sequence encompassing either position 638 of the HEXA gene, or position 181 of the HEXA gene and their respective flanking regions, wherein said nucleic acid sequence comprises the correct nucleic acid in said positions, e.g. adenine at position 638 and cytosine at position 181; (b) transfecting patients cells with said corrective vectors either ex vivo or in vivo under conditions allowing homologous recombination and expression of a correct, normal HEXA protein in sufficient quantities to reverse the disease condition.

The invention also concerns the corrective vectors described above, as well as their use for treating Tay-Sachs disease, and for preparing pharmaceutical compositions for the treatment of the disease. In another aspect the invention concerns pharmaceutical composition for the treatment of Tay-Sachs disease comprising the above-described corrective vectors.

### BRIEF DESCRIPTION OF THE DRAWINGS

The patent application file contains at least one drawing photograph executed in color. Copies of this patent application publication with color drawing photographs will be provided by the Office upon request and payment of the necessary fee.

In order to understand the invention and to see how it may be carried out in practice, a preferred embodiment will now be described, by way of non-limiting example only, with reference to the accompanying drawings, in which:

FIG. 1 is a photograph demonstrating immunofluorescence detection of a subunit in transfected COS-7 cells. COS-7 cells were transiently transfected with either pcDNA 3 α (a), pcDNA 638αA>C (b) or pcDNA3 (c). Bright fluorescence marks the subunit localization. Nucleic acid staining was performed using Propidium Iodide.

### DETAILED DESCRIPTION OF EXEMPLARY EMBODIMENTS

Definitions:

“Screening” as used herein means examining a subject to identify the presence of a mutation associated with Tay-Sachs disease. Screening also pertains to mass examination of the population or of people at risk and is particularly useful for identifying carrier status to allow informed genetic counseling.

“Tay Sachs disease” and “G_{M2} gangliosidosis type B” are used interchangeably and mean an inherited autosomal recessive disorder caused by mutations in the α chain of the A form of β-hexosaminidase (HEXA). As used herein Tay-Sachs disease (TSD), includes the severe-infantile form of G_{M2} gangliosidosis type B, as well as the milder juvenile and the late onset forms of TSD (LOTS).

“Mutant hexosaminidase A gene” as used herein means the HEXA gene, wherein at least one nucleic acid substitution, and/or deletion and/or insertion occurs, which result in impairment of its essential function. As used herein the “normal” HEXA gene sequence refers to that sequence, which results in the normal phenotype and which is the most common haplotype found in the population.

“Genetic polymorphism” as used herein refers to the variation seen at a genetic locus wherein two or more different nucleotide sequences can coexist at the same genetic locus in the DNA. The different sequences may or may not result in disease.

“Tay-Sachs carrier” as used herein means a person whose chromosomes contain a mutant HEXA gene that may be transmitted to said person's offspring.

“Tay-Sachs patient” as used herein means a person who carries a mutant HEXA gene on both chromosomes on which the gene is carried, such that the clinical symptoms of Tay-Sachs disease are exhibited.

“Allele” as used herein means an alternative form of a gene. A single allele for each gene is inherited separately from each parent.

“Oligonucleotide primers” as used herein means a molecule comprised of more than three deoxyribonucleotides or ribonucleotides. Its exact length will depend on many factors relating to the ultimate function or use of the oligonucleotide primer including temperature, source of the primer and use of the method. The oligonucleotide primer can occur naturally as in a purified restriction digest or be produced synthetically. The oligonucleotide primer is capable of acting as a point of initiation of synthesis when placed under conditions which induce synthesis of a primer extension product complementary to a nucleic acid strand. The conditions can include the presence of nucleotides and an inducing agent such as DNA polymerase at a suitable temperature and pH. Although the primer preferably is single stranded, it may alternatively be double stranded. If it is double stranded, the primer must first be treated to separate its strands before it is used to produce extension products. In the preferred embodiment, the primer is an oligodeoxyribonucleotide. The primer must be sufficiently long to prime the synthesis of extension products in the presence of the inducing agent.

“Complementary” as used herein means a nucleic acid molecule having a sequence which complements a reference template sequence, whereby the two sequences can specifically hybridize.

“Variant” as used herein means a nucleic acid molecule encoding a mutant HEXA gene having at least 95% homology (identity) in the nucleic acid level to the mutant HEXA gene sequence of SEQ ID Nos. 1 or 2.

“Flanking” as used herein means the sequence of nucleotides adjacent, on both sides of the mutated nucleic acid (e.g. adjacent to position 638 or adjacent to position 181).

Most of the late onset Tay Sachs (LOTS) disease Ashkenazi and non-Ashkenazi patients have a common molecular scheme, harboring the 805G>A mutation responsible for the less severe form of G_{M2} gangliosidosis type B in compound heterozygosity with an infantile mutation.

This invention relates to newly identified mutations in the HEXA gene which are associated with LOTS disease. More specifically, one mutation is an A to C change at base pair position 638 of the HEXA gene, and another mutation is C to T change at base pair position 181 of said gene.

Without being limited by theory, it is assumed that both mutations alter the secondary structure of the α-precursor peptide resulting in its retention in the ER/cis Golgi Network and its subsequent proteosomal degradation.

Mis-sense mutations in the HEXA gene which affect the tertiary structure of the early α precursor in the ER/cis Golgi Network and disrupt its folding or dimerization have been previously reported. Such mutant proteins are retained in the ER/cis Golgi Network by the quality control system and are subsequently directed to proteosomal degradation and thus their routing to the lysosomes is prevented (Mahuran 1999; Hiller et al 1996). Indeed, both 638A>C and 181C>T result in non-conservative amino-acid substitutions: the 638A>C mutation results in substitution of Y to S and 181C>T results in substitution of L to S. These substitutions are likely to result in alteration of the secondary structure of the α-precursor peptide. It can be concluded that the two identified mutations are disease-causing mutations.

With knowledge of the novel mutations of the HEXA gene as disclosed herein, screening for presymptomatic homozygotes and heterozygous carriers, including prenatal diagnosis and screening can be readily carried out.

Individuals carrying mutations in the HEXA gene may be detected at either the DNA or RNA level using a variety of techniques that are well known in the art. The mutation analysis may be performed on samples of RNA by reverse transcription into cDNA.

The genomic DNA or RNA used for the diagnosis may be obtained from an individual's cells, such as those present in peripheral blood, urine, saliva, surgical specimen, and autopsy specimens. The DNA may be used directly or may be amplified in vitro through use of methods well known in the art e.g. PCR, prior to mutation analysis. Genomic HEXA may be amplified using any set of primer pairs as previously disclosed (Triggs-Raine et al 1991). As a non limiting example, cDNA may be amplified using oligonucleotide primers designated SEQ ID. Nos. 3-10, as detailed in Table 2. For the identification of HEXA 638A>C exon 6 should be amplified e.g. using primers of SEQ. ID. Nos. 5 and 6. For the amplification of 181C>T exon 1 should be amplified e.g. using primers of SEQ ID. Nos. 3 and 4.

In situ hybridization may also be used to detect the HEXA mutations of the invention.

The methodology for preparing nucleic acids in a form that is suitable for mutation detection is well known in the art. For example, suitable probes for detecting a given mutation include the nucleotide sequence at the mutation site and encompass a sufficient number of nucleotides to provide a means of differentiating a normal from a mutant allele. Any probe or combination of probes capable of detecting any one of the HEXA mutations herein described are suitable for use in this invention. Examples of suitable probes include those complementary to either the coding or non coding strand of the DNA. Similarly, suitable PCR primers are complementary to sequences flanking the mutation site. Production of these primers and probes can be carried our in accordance with anyone of the many routine methods, e.g. as disclosed in Sambrook et al. sup 45.

Probes for use with this invention should be long enough to specifically identify or amplify the relevant HEXA mutations with sufficient accuracy to be useful in evaluating the risk of an individual to be a carrier or having Tay-Sachs disease. In general, suitable probes and primers will comprise, preferably at a minimum, an oligomer of at least 16 nucleotides in length. Since calculations for mammalian genomes indicate that for an oligonucleotide 16 nucleotides in length, there is only one chance in ten that a typical cDNA library will fortuitously contain a sequence that exactly matches the sequence of a nucleotide. Therefore, suitable probes and primers are preferably 18 nucleotides long, which is the next larger oligonucleotide fully encoding an amino acid sequence (i.e., 6 amino acids in length).

By use of nucleotide sequences provided by this invention, effective and accurate testing procedures are made available to identify carriers of mutant 638A>C and 181C>T alleles of HEXA, as well as pre- and postnatal diagnosis of fetuses, children and adults carrying either one or two mutant alleles. These testing procedures should be combined with assays directed towards the identification of other Tay-Sachs mutations known in the art and together will generate a more accurate screening tool that allows the identification of a broad array of carriers and patients which were previously not identified.

Many versions of conventional genetic screening tests are known in the art. Several examples are U.S. Pat. No. 5,217,865 describing screening for Tay-Sachs disease, WO 91/02796 for cystic fibrosis, U.S. Pat. No. 5,227,292 for neurofibromatosis, WO 93/06244 for Gaucher disease and WO 02/059381 for familial dysautonomia. Thus, in accordance with the state of the art regarding assays for such genetic disorders, several types of assays are conventionally employed for mutation detection. For example, the detection of the 638A>C or 181C>T mutations in the HEXA gene sequence can be accomplished by a variety of methods including but not limited to, DNA sequencing, restriction-fragment-length-polymorphism (RFLP) analysis based on allele-specific restriction-endonuclease cleavage, hybridization with allele-specific oligonucleotide probes including immobilized oligonucleotides, oligonucleotide or GeneChip arrays, peptide nucleic acid (PNA) probes, methylation-specific PCR, pyrosequencing analysis, acycloprime analysis, reverse dot blot, TaqMan, Molecular Beacons, Intercalating dye, FRET primers, AlphaScreen, SNPstream, genetic bit analysis, Multiplex minisequencing, SNaPsht, MassEXTEND, MassArray, GOOD assay, Microarray miniseq, arrayed primer extension, Microarray primer extension, Tag arrays, Coded microspheres, template-directed incorporation (TDI), fluorescence polarization, Colorimetric oligonucleotide ligation assay (OLA), Sequence-coded OLA, Microarray ligation, Ligase chain reaction, Padlock probes, Rolling circle amplification, Invader assay, MLPA and MS-MLPA.

The identification of the novel HEXA mutations of the invention also has therapeutic implications. Indeed, one aspect of the present invention is a therapy which circumvents or overcomes the defect in the HEXA gene caused by the 638A>C or 181C>T mutations via a gene therapy approach based on site-directed mutagenesis.

Gene therapy, utilizing recombinant DNA technology to deliver specifically designed corrective vectors into patient cells, will supply the patient with a normal, corrected gene product in vivo. Accordingly, a recombinant vector is designed comprising a nucleic acid sequence encompassing position 638 of the HEXA gene, or position 181 of the HEXA gene and their respective flanking regions, wherein said nucleic acid sequence comprises the correct nucleic acid, e.g. adenine at position 638 and cytosine at position 181. In gene therapy of Tay-Sachs disease, the corrective vector is delivered to the affected individual in a form and amount such that homologous recombination occurs and the correct gene is expressed and will translate into sufficient quantities of HEXA protein to reverse the effects of the mutated HEXA gene. Current approaches to gene therapy include viral vectors, cell-based delivery systems and delivery agents. Further, ex vivo gene therapy could also be useful. In ex vivo gene therapy, cells (either autologous or otherwise) are transfected with the corrective vector and implanted or otherwise delivered into the patient. Such cells thereafter express the normal HEXA gene product in vivo and would be expected to assists a patient with Tay-Sachs disease. Ex vivo gene therapy is described in U.S. Pat. No. 5,399,346.

Appropriate vectors include retroviruses, adenovirus, adeno associated virus (AAV), vaccinia virus, bovine papilloma virus or members of the herpes virus group such as Epstein-Barr virus. The viral vectors are preferably replication deficient. Non viral methods of in vivo delivery are also suitable, e.g. liposomes.

Materials and Methods

Description of Patients which Participated in the Present Study

Patient I: A 30-year-old woman, born in Russia to unrelated parents and with no illnesses worthy of note in her family. She was in good health until age 15, when she started having difficulties in jumping, walking and coordination. Her symptoms worsened rapidly within three years. She had emotional lability and was treated by a psychiatrist under the diagnosis of hysterical bursts. At age 24 she was diagnosed as having ‘ALS’. Since then she had more difficulties walking and her speech has become unclear. On examination she had dysarthric speech with nasal quality. Cranial nerves were intact. Upper limbs showed normal strength and muscle mass. There was severe atrophy of thigh muscles. Strength of iliopsoas, quadriceps, and hamstrings was 2-3/5 (MRC scale) bilaterally. Other lower limbs muscles were normal. Deep tendon reflexes were normal. She had pronounced dysmetria on finger-nose test. Her gait was waddling, and she used Gower's maneuver to get up from sitting. MRI showed severe cerebellar atrophy with normal structure of the cerebral hemispheres. EMG revealed severe, chronic nonactive neurogenic changes in the lower limbs muscles.

To summarize, the clinical features were of cerebellar ataxia and lower motor neuron signs of the lower limbs.

Patient II: This 67-year-old Ashkenazi Jew was a mother of two children who were diagnosed as LOTS: the son had an unusually slow ALS-like syndrome and the daughter had a diffuse neurodegenerative disease affecting mentation, cerebellum, motor neurons, and pyramidal system. She noticed tremor of the hands (more on the left) after the delivery of her first daughter at age 30. The tremor worsened with years and she needed someone to assist her with eating and dressing and lost her ability to write. She did not report any weakness of the hands and was still walking six km every day. There were no sensory or cognitive symptoms. Examination revealed mild, slow rhythmic tremor of the head and tongue at rest. A marked coarse tremor of both hands (more on the left) was noted at rest and this tremor was aggravated upon action and became flapping-like. There was no weakness but the muscle tone was markedly reduced in the upper limbs and slightly reduced in the lower. Tendon reflexes were elicited and there were no pyramidal signs. The patient could not perform repeated changing movements of her hands as the tremor became so marked that she needed to hold one hand with the other. The tremor was also aggravated at walking but her gait was otherwise normal. She did have minimal impairment of the shin to heal test. She did not show any cognitive or psychiatric decline and did not have any signs of neurological involvement of other systems until death at age 83.

Thus, this patient had a very atypical syndrome of rubro-cerebellar tremor without other neurological phenomena.

The patients were tested for Hex A activities in serum and leukocytes either by the heat inactivation method using 4-methylumbelliferyl 2-acetoamido-2-deoxy-β D-glucopyranoside (4-MUG) or by 4-methylumbelliferyl-N-acetyl-β-D-glucosaminide-6-sulfo (4-MUGS) as a substrate (Fuchs et al 1983; Inui and Wenger 1984). All patients were shown to have severe Hex A deficiency in the range known for patients suffering from G_{M2} gangliosidosis type B.

Nucleic Acid Analysis

Genomic DNA was isolated from whole blood and from cultured fibroblasts using the Puregene DNA isolation kit (Gentra Systems, Inc., Minneapolis, Minn.).

Total RNA was isolated from fibroblasts using TriPure™ reagent (Roche Diagnostics, Basel, Switzerland) and mRNA was reverse transcribed using synthetic oligo-d(T) and Superscript II RNAse H-Reverse Transcriptase (Gibco Invitrogen Corporation, Carlsbad, Calif.).

PCR reactions were performed with Taq Polymerase (Bioline, Boston, Mass.).

Sequencing of PCR products from patients' cDNA and genomic DNA was performed using 373A DNA sequencing system, Applied Biosystems, Foster City, Calif.

Expression in COS-7 Cell Line

The different identified mutations were introduced into wild-type pcDNA3α vector (the α cDNA in pBluescript II KS vector was kindly provided by Dr. R. L. Proia and subcloned in pcDNA3 vector using Xhol/Notl sites) using either the QuickChange™ Site-Directed Mutagenesis kit (Stratagene, La Jolla, Calif.) (638A>C) or the Transformer™ Site Directed Mutagenesis Kit, 2^{nd} version (Clontech, Palo Alto, Calif.) (181C>T).

TABLE 1

Primer pairs for mutagenesis
(mutated nucleotides are in bold)

| | |
|---|---|
| Primer | Sequence |

| | |
|---|---|
| 638A > C sense | 5′GATCCTTCCTTCCCATCTGAGAGCTTCACT |
| | TTT3′ |
| 638A > C antisense | 5′AAAAGTGAAGCTCTCAGATGGGAAGGAAGG |
| | ATC3′ |
| 181C > T Selection | 5′CGATATCAACGTTATCGATACCGTCG3′ |
| primer | |
| 181C > T Mutagenic | 5′GCTGCTCAGTCTTCGACGAGGCC3′ |
| primer | |

Briefly, the COS-7 cell-line was grown in Dulbecco's modified Eagle's medium, supplemented with 20% fetal calf serum (FCS), antibiotics and L-glutamin (Biological Industries, Beit Haemek, Israel). Next, the cells were transiently transfected with the different cDNA constructs in Opti-MEM I Reduced Medium (Gibco Invitrogen Corporation, Carlsbad, Calif.) using FuGene6 reagent (Roche Diagnostics, Basel, Switzerland) according to the manufacturer's instructions. Finally, the cells were harvested 48 h post transfection.

Hex S Enzymatic Activities and Protein Levels

Protein concentrations were determined by the Bradford method (BioRad Laboratories, Inc., Hercules, Calif.).

For the enzyme assay, the harvested cells were re-suspended in 0.05M sodium citrate buffer, pH 4.2, and subjected to five rounds of freezing and thawing. The cell extracts were assayed for Hex S activity using 4-MUGS (Sigma, Ronkonkoma, N.Y.) specific for the catalytic activity of the α subunit (Fuchs et al 1983; Inui and Wenger 1984). All transfection experiments were performed in duplicate and enzymatic reactions in triplicate to ensure reliability of the results.

For Western blotting, the harvested cells were lysed for 1 h in a lysis buffer containing NP-40 and Complete Protease Inhibitor Cocktail (Roche Diagnostics, Basel, Switzerland). Equal amounts of total proteins from transfected COS-7 cells extracts (2.5 μg) were separated by 10% SDS-PAGE and transferred to nitrocellulose membranes (Laemmli 1970). The membranes were blocked in 5% BSA and then incubated overnight at 4° C. with 1:800 dilution of rabbit anti human Hex A IgG (kindly provided by Dr. R. A. Gravel) and 1 h with 1:25,000 dilution of Horseradish Peroxidase conjugated donkey anti-rabbit IgG (Jackson Immunoresearch). Finally, the membranes were developed using Chemiluminescent Detection System (ECL, Santa Cruz Biotechnology).

Immunolocalization in Intracellular Compartments

COS-7 cells were seeded onto cover slips at low-density 24 h post transfection. Following 24 h incubation, the cells were fixed and gently permeabilized with cold methanol at −20° C. for 30 min, blocked with 1% BSA, and incubated overnight with a 1:700 dilution of rabbit anti-human Hex A IgG at 4° C. The cells were then incubated with Fluorescein (FITC) conjugated AffiniPure goat anti-rabbit IgG (green fluorescence) (Jackson ImmunoResearch Laboratories, Inc., West Grove, Pa.) diluted 1:75 for 1 h and then treated with the nucleic acid marker Propidium Iodide (red fluorescence) (Sigma, Ronkonkoma, N.Y.) for 1 h. A confocal laser-scanning microscope (LSM 410 invert, Carl Zeiss, Oberkochen, Germany) equipped with a 25-mW krypton-argon laser (488 and 568 maximum lines) was used to analyze the slides.

### EXAMPLE 1

### Identification of Mutations in the HEXA Gene

Genomic DNA was isolated from the whole blood of patient I and from cultured fibroblasts of patient II. In addition, mRNA was isolated from fibroblast and reverse transcribed to cDNA

The cDNA was amplified using primer pairs as described in Table 2. Primer pairs used for amplifying genomic HEXA (exons and their flanking introns) were as described before (Triggs-Raine et al 1991).

TABLE 2
Primers used for HEXA cDNA amplification

| SEQ | | |
|---|---|---|
| ID | | |
| NO. | Primer | Sequence |
| 3 | Exon1-5 forward | TTCCAAACCTCCGACCAGCGC |
| 4 | Exon1-5 reverse | CAGAGTGTCCAGGATTGCTAG |
| 5 | Exon5-8 forward | TTCTTTATCAACAAGACTGAGATTG |
| 6 | Exon5-8 reverse | GCAGGTGAAATCAACCTCATCTCCTCC |
| 7 | Exon8-11 forward | ATCCCTGGATTACTGACTCCTTGCTAC |
| 8 | Exon8-11 reverse | CTTCAAATGCCAGGGGTTCCA |
| 9 | Exon11-14 forward | AGCCAGACACAATCATACAG |
| 10 | Exon11-14 reverse | CCTTTCTCTCCAAGCACAGG |

Genomic DNA from the patients was first screened for some common mutations: 1278insTATC, IVS9+1G>A, IVS12+1G>C and 805G>A (Myerowitz et al 1988; Akerman et al 1992; Ohno and Suzuki 1988; Navon and Proia 1989; Paw et al 1989). Both patients were found to carry the common 805G>A (G269S) mutation. Sequencing the amplified cDNA and genomic HEXA of the patients revealed that they are both compound heterozygotes. They both carried the common 805G>A (G269S) mutation, but each carried an additional point mutation: a novel 638A>C transversion (Y213S) in exon 6 in patient I, and a novel 181C>T transition (L61S) in exon 1 in patient II. The patients' ethnicity and genotype are summarized in Table 3.

TABLE 3
The patients - clinical and molecular summary

| | | Mutation: | Mutation: |
|---|---|---|---|
| Patient No. | Ethnicity | Allele I | Allele II |
| I | Russia | 805 G > A | 638 A > C |
| | | aa G269S | aa Y213S |
| II | Israel | 805 G > A | 181 C > T |
| | Ashkenazi | aa G269S | aa L61S |

The mutations, 638A>C, and 181C>T were confirmed by restriction analyses with NdeI, and MboII. The 638A>C mutations abolishes a restriction site for NdeI, and the 181C>T mutation creates a restriction site for MboII respectively.

### EXAMPLE 2

### Enzymatic Activities of the Mutant Proteins and Levels of the Mutant α Subunits in Transfected COS-7 Cells

To examine the role of the different identified mutations in the pathology of the disease, we performed expression studies. Transfections with the wild-type construct pcDNA3α resulted in ˜8.5-fold increase in Hex S activity as compared to the background activity of the mock-transfected cells. Transfections with the mutant constructs pcDNA3α638A>C and pcDNA3α181C>T all resulted in reduced Hex S activities, which were virtually indistinguishable from one another and from the background activity of the mock-transfected cells. Results are summarized in Table 4.

The reduction in Hex S activity obtained following transfections with the mutant constructs could result from either an inactive enzyme in the lysosomes or a defect in the synthesizing route of the protein. The α subunit protein level in COS-7 transfected with pcDNA3α638A>C cells was evaluated by Western blotting yielding an undetectable level of the mature α subunit (data not shown). This ruled out the possibility of an inactive enzyme.

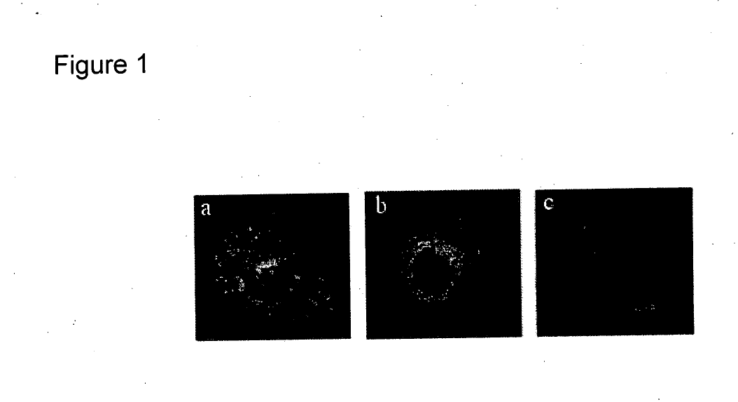

Direct immunofluorescence was performed to determine the fate of the mutant proteins in COS-7 cells (FIG. 1). Following transfections with the wild-type construct, a punctuate staining was obtained, indicative of endosomal-lysosomal localization of the expressed α subunit (Van Dongen et al 1984). In contrast, transfection with the mutant construct pcDNA3α638A>C led to an intensely perinuclear staining with diffusion into the cytoplasm, indicating unprocessed α subunits accumulating in the ER (Hoefsloot et al 1990) no mature α subunit could be detected.

GOR-IV software (Garnier et al 1996) was used to predict the possible consequences of the identified mutations on the secondary structure of the precursor α subunit.

TABLE 4
Enzymatic activities of the mutant proteins expressed in COS-7 cell line measured
with the 4-MUGS substrate
The results shown in table 4 represent the average of two different experiments
measuring expression in triplicate.

| | Hex S | | | | 2^{nd} |
|---|---|---|---|---|---|
| | activity | Hex S | | | structure |
| cDNA | (units*/□g | activity** | Mature α | α subunit | alteration |
| construct | protein) | (%) | level | immunolocalization | (GOR-IV) |
| pc DNA3α | 2.86 ± 0.9 | 100 | detectable | Endosomal- | |
| (wild type) | | | | lysosomal | |
| pcDNA3α638A > C | 0.42 ± 0.14 | 3.6 | undetectable | Perinuclear | + |
| pcDNA3 (mock) | 0.33 ± 0.08 | 0 | undetectable | undetectable | |
| pcDNA3α181C > T | 0.57 ± 0.16*** | 9.4*** | NA | NA | + |

*One unit is defined as the activity that releases 1 nmol of 4-methylumbeliferone per hour.
**Values were calculated by subtracting the mock transfection background activity and are given as the percentage of the wild-type transfection.
***Was performed in a separate experiment. Calculation of Hex S activity was performed according to the Hex S of the wild type and mock in that experiment.

Taken together, the results confirm the involvement of the newly identified mutations in Tay-Sachs disease pathology (summarized in Table 5)

TABLE 5
Confirmation of disease-causing mutations

| | | | | 2^{nd} |
|---|---|---|---|---|
| | Expression in COS-7 cell-line | | | structure |
| | Hex S | Mature α | α subunit | alteration |
| Mutation | activity | level | Immunolocalization | (GOR-IV) |
| 638A > C | Deficient | Undetectable | perinuclear | + |
| 181C > T | Deficient | NA | NA | + |

NA - not performed

Patient II (HEXA 181C>T) is unique with respect to her phenotype, age of onset and expression of very mild symptoms, which were not observed in other Ashkenazi LOTS patients. This can perhaps be explained by her different genotype, seen for the first time, since all Ashkenazi LOTS patients reported to date harbor the 805G>A (G269S) in compound heterozygosity with either 1278+TATC or IVS12+1G>A. Interestingly, the very mild unique manifestation displayed by this patient implies that 181C>T may enable residual Hex A activity.

### REFERENCES

Akerman B R, Zielenski J, Triggs-Raine B L et al (1992) A mutation common in non-Jewish Tay-Sachs disease: frequency and RNA studies. Hum Mutat 1: 303-309.
Argov Z, Navon R (1984) Clinical and genetic variations in the syndrome of adult G_{M2} gangliosidosis resulting from hexosaminidase A deficiency. Ann Neurol 16: 14-20
Conzelmann E, Kytzia H J, Navon R, Sandhoff K (1983) Ganglioside G_{M2} N-acetyl-β-D-galactosaminidase activity in cultured fibroblasts of late-infantile and adult G_{M2} gangliosidosis patients and of healthy probands with low hexosaminidase level. Am J Hum Genet 35: 900-913.
Conzelmann E, Sandhoff K (1983-4) Partial enzyme deficiencies: residual activities and the development of neurological disorders. Dev Neurosci 6: 58-71.
Fuchs W, Navon R, Kaback M M, Kresse H (1983) Tay-Sachs disease: one-step assay of β-N-acetylhexosaminidase in serum with a sulphated chromogenic substrate. Clin Chim Acta 133: 253-261.
Garnier J, Gibrat J F, Robson B (1996) GOR secondary structure prediction method version IV. Methods Enzymol 266: 540-553.
Gravel R A, Kaback M M, Proia R L, Sandhoff K, Suzuki K, Suzuki K (2001) The G_{M2} Gangliosidoses. In Scriver C R, Beaudet A L, Sly W S, Valle D, eds: Childs B, Kinzler K W, Vogelstein B, assoc. eds. The Metabolic and Molecular Basis of Inherited Diseases, 8^{th} edn. New York: McGraw-Hill, 3827-3876.
Hiller M M, Finger A, Schweiger M, Wolf D H (1996) ER degradation of a misfolded luminal protein by the cytosolic ubiquitin-proteasome pathway. Science 273: 1725-1728.
Hoefsloot L H, Willemsen R, Kroos A M et al (1990) Expression and routeing of human lysosomal a-glucosidase in transiently transfected mammalian cells. Biochem J 272: 485-492.
Inui K, Wenger D A (1984) Usefulness of 4-methylumbelliferyl-6-sulfo-2-acetamido-2-deoxy-β-D-glucopyrano side for the diagnosis of G_{M2} gangliosidoses in leukocytes. Clin Genet 26: 318-321.
Laemmli U K (1970) Cleavage of structural proteins during the assembly of the head of bacteriophage T4. Nature 227: 680-685.
Mahuran D J (1999) Biochemical consequences of mutations causing the G_{M2} gangliosidoses. Biochim Biophys Acta 1455: 105-138.
Myerowitz R, Costigan F C (1988) The major defect in Ashkenazi Jews with Tay-Sachs disease is an insertion in the gene for the a-chain of β-hexosaminidase. J Biol Chem 263: 18587-18589.
Myerowitz R (1997) Tay-Sachs disease-causing mutations and neutral polymorphisms in the Hex A gene. Hum Mutat 9: 195-208.
Navon R (1991) Molecular and clinical heterogeneity of adult G_{M2} gangliosidosis. Dev Neurosci 13: 295-298.
Navon R, Proia R L (1989) The mutations in Ashkenazi Jews with adult G_{M2} gangliosidosis, the adult form of Tay-Sachs disease. Science 243: 1471-1474.
Ohno K, Suzuki K (1988) A splicing defect due to an exon-intron junctional mutation results in abnormal β-hexosaminidase a chain mRNAs in Ashkenazi Jewish patients with Tay-Sachs disease. Biochem Biophys Res Commun 153: 463-469.
Paw B H, Kaback M M, Neufeld E F (1989) Molecular basis of adult-onset and chronic G_{M2} gangliosidoses in patients of Ashkenazi Jewish origin: substitution of serine for glycine at position 269 of the a-subunit of β-hexosaminidase. Proc Natl Acad Sci USA 7: 2413-2417.
Triggs-Raine B L, Akerman B R, Clarke J T, Gravel R A (1991) Sequence of DNA flanking the exons of the HEXA gene, and identification of mutations in Tay-Sachs disease. Am J Hum Genet 49: 1041-1054.
Van Dongen J M, Barneveld R A, Geuze H J, Galjaard H (1984) Immunocytochemistry of lysosomal hydrolases and their precursor forms in normal and mutant human cells. Histochem J16: 941-954.

1. A method of screening a subject to determine if said subject has a mutation associated with Tay Sachs disease, comprising:
a. Providing a biological sample containing nucleic acid molecules of the subject to be screened;
b. Detecting a 638A>C or 181C>T mutation in the α chain of β-hexosaminidase (hereinafter HEXA) gene in said biological sample.

2. A method according to claim 1, wherein said nucleic acid molecules comprise genomic DNA, RNA or cDNA.

3. A method according to claim 1, wherein said genetic material is amplified prior to detection of said mutations.

4. A method according to claim 3, wherein said genetic material is amplified using HEXA specific oligonucleotide primers.

5. A method according to claim 4, wherein said oligonucleotide primers are selected from the group consisting of SEQ ID Nos. 3-6.

6. A method according to claim 1, wherein the mutation is detected by restriction fragment length polymorphism analysis.

7. A method according to claim 6 wherein said restriction fragment length polymorphism analysis is performed using a NdeI restriction enzyme; and wherein the presence of the 638A>C HEXA mutation is detected by the abolishment of the NdeI restriction site.

8. A method according to claim 6 wherein said restriction fragment length polymorphism analysis is performed using a MboII restriction enzyme; and wherein the presence of the 181C>T HEXA mutation is detected by the creation of a MboII restriction site.

9. A method according to claim 1, wherein the mutation is detected by an allele specific oligonucleotide hybridization assay.

10. A method according to claim 9, wherein the hybridization assay is accomplished using probes, at least 18 nucleic acids long, that span the HEXA 638A>C mutation.

11. A method according to claim 9, wherein the hybridization assay is accomplished using probes, at least 18 nucleic acids long, that span the HEXA 181C>T mutation.

12. A method according to claim 10, wherein the hybridization is accomplished using a probe comprising the nucleotide sequence 5 ′AAAAGTGAAGCTCTCAGATGGGAAGGAAGGATC3′.

13. A method according to claim 11, wherein the hybridization is accomplished using a probe comprising the nucleotide sequence 5 ′CTCGTCGAAGACTGAGCA3′.

14. An oligonucleotide probe for detecting a HEXA 638A>C or 181C>T mutation, selected from the group consisting of:
a. A nucleic acid molecule comprising a nucleic acid sequence complementary to the region flanking position 638 of the HEXA gene;
and wherein the nucleic acid complementing position 638 is Guanine;
b. A nucleic acid molecule comprising a nucleic acid sequence complementary to the region flanking position 181 of the HEXA gene; and wherein the nucleic acid complementing position 181 is Adenine;
c. A nucleic acid molecule comprising the nucleotide sequence 5′AAAAGTGAAGCTCTCAGATGGGAAGGAAGGATC3′; and
d. A nucleic acid molecule comprising the nucleotide sequence 5 ′CTCGTCGAAGACTGAGCA3′.

15. The oligonucleotide probe according to claim 14 labeled with a detectable marker.

16. A kit for assaying for the presence of a HEXA mutation in an individual comprising at least one oligonucleotide probe capable of detecting the HEXA 638A>C or 181C>T mutation in accordance with claim 15.

17. A kit according to claim 16, further comprising primers capable of amplifying the region containing said mutations.

18. A kit according to claim 17, wherein said primers are selected from the group consisting of SEQ ID Nos. 3-10.

19. A kit according to claim 16, further comprising an oligonucleotide probe which specifically hybridizes to one or more additional HEXA mutations wherein said additional mutations are associated with Tay-Sachs disease.

20. A kit according to claim 19 wherein said additional HEXA mutations are selected from the group consisting of 1278insTATC, IVS9+1G>A, IVS12+1G>C and 805G>A.

21. A kit according to claim 16, further comprising an oligonucleotide probe which specifically hybridizes to one or more additional mutant genes, wherein said additional gene codes for a protein associated with an additional genetic disease or disorder.

22. A kit according to claim 21 wherein said additional genetic disease or disorder is selected from the group consisting of: Canavan's disease, Familial dysautonomia, Gaucher, Cystic Fibrosis, Fanconi anemia and Bloom syndrome.

23. A method according to claim 1, further comprising a determination of whether said individual is homozygous or heterozygous for said mutation.

24. A method according to claim 1, further comprising a determination of whether said individual is a Tay-Sachs disease carrier or a Tay-Sachs disease patient.

25. An isolated nucleic acid molecule encoding a mutant α chain of β-hexosaminidase (hereinafter HEXA) wherein the Adenine nucleotide at position 638 is replaced with Cytosine; and fragments thereof comprising position 638 and its flanking regions.

26. An isolated nucleic acid molecule encoding a mutant α chain of β-hexosaminidase (hereinafter HEXA) wherein the Cytosine nucleotide at position 181 is replaced with Thymine; and fragments thereof comprising position 181 and its flanking regions.

27. An isolated nucleic acid molecule according to claim 25, wherein the nucleic acid molecule comprises the sequence designated SEQ ID No. 1 or a variant thereof having at least 95% homology.

28. An isolated nucleic acid molecule according to claim 26, wherein the nucleic acid molecule comprises the sequence designated SEQ. ID No. 2 or a variant thereof having at least 95% homology.

29. An isolated mutant HEXA polypeptide encoded by a nucleic acid sequence according to claim 25.

30. A recombinant vector comprising the nucleic acid molecules according to claim 25.

31. A recombinant vector according to claim 30, wherein said nucleic acid molecule is operably linked to an expression control sequence suitable for expression of said nucleic acid sequence in a host cell.

32. A host cell comprising the recombinant vector according to claim 31.

33. A method of producing a mutant HEXA polypeptide, comprising:
a. Culturing a host cell according to claim 32 in a cell culture medium under conditions whereby the mutant HEXA is expressed; and
b. Isolating said mutant HEXA polypeptide.

34. A method of screening or diagnosing patients or carriers of Tay Sachs disease in the Ashkenazi Jewish population comprising detecting a HEXA 181C>T mutation in said patients' genetic material.

35. A method of treating Tay-Sachs disease by gene therapy comprising:
a. providing corrective recombinant vectors comprising a nucleic acid sequence encompassing position 638 of the HEXA gene and its respective flanking regions; wherein said nucleic acid sequence comprises adenine at position 638; and
b. transfecting cells of a Tay-Sachs patient with said corrective vectors ex vivo or in vivo under conditions allowing homologous recombination and expression of a correct HEXA protein in sufficient quantities to reverse the disease condition.

36. A method of treating Tay-Sachs disease by gene therapy comprising:
a. providing corrective recombinant vectors comprising a nucleic acid sequence encompassing position 181 of the HEXA gene and its respective flanking regions; wherein said nucleic acid sequence comprises cytosine at position 181; and
b. transfecting cells of a Tay-Sachs patients with said corrective vectors ex vivo or in vivo under conditions allowing homologous recombination and expression of a correct HEXA protein in sufficient quantities to reverse the disease condition.

37. A corrective recombinant vector comprising a nucleic acid sequence encompassing position 638 of the HEXA gene and its respective flanking regions; wherein said nucleic acid sequence comprises adenine at position 638.

38. A corrective recombinant vector comprising a nucleic acid sequence encompassing position 181 of the HEXA gene and its respective flanking regions; wherein said nucleic acid sequence comprises cytosine at position 181.

39. Use of a vector according to claim 37 for preparing pharmaceutical compositions for the treatment of Tay-Sachs disease.

40. A pharmaceutical composition for the treatment of Tay-Sachs disease comprising a corrective vector according to claim 37.
